(19) United States
(12) Patent Application Publication
(10) Pub. No.: US 2015/0313447 A1
(43) Pub. Date: Nov. 5, 2015

(54) INSERTION INSTRUMENT AND ENDOSCOPE

(71) Applicant: OLYMPUS CORPORATION, Tokyo (JP)

(72) Inventor: Keiichi ARAI, Tokyo (JP)

(73) Assignee: OLYMPUS CORPORATION, Tokyo (JP)

(21) Appl. No.: 14/796,103

(22) Filed: Jul. 10, 2015

Related U.S. Application Data
(63) Continuation of application No. PCT/JP2014/070617, filed on Aug. 5, 2014.

(30) Foreign Application Priority Data
Aug. 28, 2013 (JP) 2013-176968

(51) Int. Cl. A61B 1/005

(52) U.S. Cl. CPC A61B 1/0052; A61B 1/0057

(57) ABSTRACT

An insertion instrument includes an insertion portion, an operation portion connected to the insertion portion, a first bending region and a second bending region on a distal end side of the insertion portion, a bending wire that is inserted through the inside of the insertion portion and the operation portion and that causes the bending portion to perform a bending operation, a wire movement restriction portion that restricts the bending wire, a bending state switching mechanism that switches between a first bending state in which the first bending region and the second bending region perform a bending operation and a second bending state in which only the first bending region performs a bending operation, and a wire movement restriction portion switching mechanism that switches the wire movement restriction portion in synchrony with an operation that switches to the first bending state or the second bending state.

### CROSS REFERENCE TO RELATED APPLICATION

This application is a continuation application of PCT/JP2014/070617 filed on Aug. 5, 2014 and claims benefit of Japanese Application No. 2013-176968 filed in Japan on Aug. 28, 2013, the entire contents of which are incorporated herein by this reference.

### BACKGROUND OF THE INVENTION

1. Field of the Invention

The present invention relates to an insertion instrument and an endoscope that include a bending portion in an insertion portion, in which the bending portion is switchable between a first bending state and a second bending state which have mutually different bending radii.

2. Description of the Related Art

In a medical field, endoscopes are utilized that can carry out observation or various kinds of therapeutic treatment by inserting an elongated insertion portion into the body. In an endoscope, an observation optical system for picking up an observation image of an observation site is provided in a distal end portion of the elongated insertion portion that is inserted into the body. Further, a bending portion is provided on a distal end side of the insertion portion of the endoscope to facilitate insertion as far as a deep part within the body and also to make it possible to turn the observation optical system of the distal end portion in a desired direction.

In the bending portion, for example, a plurality of bending pieces are rotatably connected consecutively, and the bending portion is configured so as to bend in the two directions of upward and downward, or the four directions of upward, downward, left and right. The bending portion is configured so as to, for example, perform a bending operation when a rotary knob that is a bending operation apparatus provided in an operation portion is rotated.

According to an endoscope that realizes a reduced diameter with respect to a bending shape, it is possible to bend a bending portion by 180° or by more than 180° inside the stomach or the colon or the like to perform rearward observation (also referred to as “inverted observation”) in which observation is performed in the direction opposite to the insertion direction of the insertion portion.

In this regard, in an endoscope for observing the colon, when performing a technique that bends the bending portion and lodges the distal end portion side of the insertion portion in tissue inside the body cavity and draws in and linearizes the colon, there is a concern that the lodgment of the distal end portion side of the insertion portion in the tissue will be weakened by making the bending shape a small diameter and thus linearization will be difficult.

To solve the aforementioned problem, Japanese Patent No. 4856289 discloses an insertion instrument and an endoscope having a configuration that, with a single bending portion, can vary the bending length while also having a simple configuration.

In an endoscope 100 as an insertion instrument that is shown in FIG. 1 of the aforementioned patent publication, as shown in FIG. 1A, when a proximal end portion of an inside guide sheath 101 is placed in a fixed state by means of a link mechanism portion 102, when a bending knob 103 is operated to pull a bending wire 104, only a first bending region 105a that constitutes a distal end side of the bending portion 105 bends in a manner that takes a distal end 101a of the inside guide sheath 101 as the origin.

On the other hand, as shown in FIG. 1B, when the fixed state is released by the link mechanism portion 102, the proximal end portion of the inside guide sheath 101 enters a non-fixed state. In the non-fixed state, upon the bending wire 104 being pulled, the entire bending portion 105 bends in a manner that takes a distal end 106a of an outside guide sheath 106 as the origin. The bending portion 105 includes the first bending region 105a on the distal end side and a second bending region 105b that is connected to the first bending region 105a.

According to the technology disclosed in Japanese Patent No. 4856289, because the link mechanism portion 102 is configured so as to be capable of switching between a fixing state and a non-fixing state by operation of a fixing switching member 108, while employing a single bending portion it is possible to selectively obtain a short bending state in which a bending radius at which only one part of the distal end side of the bending portion 101 bends is small, and a long bending state in which the bending radius at which the entire bending portion bends is large in comparison to the short bending state.

### SUMMARY OF THE INVENTION

An insertion instrument according to one aspect of the present invention includes: an elongated insertion portion to be inserted into a subject; a bending portion that is bendably provided on a distal end side of the insertion portion and that has a first bending region and a second bending region; a wire that is inserted through inside of the insertion portion and that is capable of performing an advancing movement and a retreating movement along the insertion portion to cause the bending portion to perform a bending operation; a bending state switching mechanism that switches between a first bending state in which the first bending region and the second bending region of the bending portion perform a bending operation and a second bending state in which only the first bending region performs a bending operation; a restriction portion that restricts a range in which the wire moves in the first bending state or the second bending state; and a restriction portion switching mechanism that, in response to an operation that switches to the first bending state or the second bending state by the bending state switching mechanism, switches a range in which the wire moves by means of the restriction portion.

An endoscope according to one aspect of the present invention is equipped with a configuration of an insertion instrument that includes: an elongated insertion portion to be inserted into a subject; a bending portion that is bendably provided on a distal end side of the insertion portion and that has a first bending region and a second bending region; a wire that is inserted through inside of the insertion portion and that is capable of performing an advancing movement and a retreating movement along the insertion portion to cause the bending portion to perform a bending operation; a bending state switching mechanism that switches between a first bending state in which the first bending region and the second bending region of the bending portion perform a bending operation and a second bending state in which only the first bending region performs a bending operation; a restriction portion that restricts a range in which the wire moves in the first bending state or the second bending state; and a restriction portion switching mechanism that, in response to an operation that switches to the first bending state or the second bending state by the bending state switching mechanism, switches a range in which the wire moves by means of the restriction portion.

### BRIEF DESCRIPTION OF THE DRAWINGS

FIG. 1A is a view illustrating an endoscope that has a single bending portion, in which a short bending state in which only a first region of the bending portion is caused to perform a bending operation and in which the bending radius is small and a long bending state in which the entire bending portion that includes the first region and a second region of the bending portion are caused to perform a bending operation and in which the bending radius is large in comparison to the short bending state are selectively obtained;

FIG. 1B is a view illustrating an endoscope that has a single bending portion, in which a short bending state in which only a first region of the bending portion is caused to perforin a bending operation and in which the bending radius is small and a long bending state in which the entire bending portion that includes the first region and a second region of the bending portion are caused to perform a bending operation and in which the bending radius is large in comparison to the short bending state are selectively obtained;

FIG. 2 is a view illustrating a configuration example of an endoscope apparatus including an endoscope of the present embodiment;

FIG. 3 is a view illustrating an insertion portion and the arrangement of coils and the like of the endoscope;

FIG. 4 is a cross-sectional view along a line indicated by arrows Y4-Y4 in FIG. 3;

FIG. 5 is a view illustrating the configuration inside an operation portion of the endoscope;

FIG. 6A is a view illustrating a state in which a range of movement of a wire is defined by a fixed stopper, and a state in which a range of movement of a wire is defined by a moving stopper;

FIG. 6B is a view illustrating a state in which a range of movement of a wire is defined by a fixed stopper, and a state in which a range of movement of a wire is defined by a moving stopper;

FIG. 7A is a view that relates to another configuration example of a wire movement restriction portion switching mechanism, and illustrates a state in which a range of movement of a wire is defined by a moving stopper and a state in which a range of movement of a wire is defined by a fixed stopper;

FIG. 7B is a view that relates to another configuration example of the wire movement restriction portion switching mechanism, and illustrates a state in which a range of movement of a wire is defined by a moving stopper and a state in which a range of movement of a wire is defined by a fixed stopper;

FIG. 8A is a view that relates to an additional configuration example of the wire movement restriction portion switching mechanism, and illustrates a state in which a range of movement of a wire is defined by a moving stopper and a state in which a range of movement of a wire is defined by a fixed stopper;

FIG. 8B is a view that relates to an additional configuration example of the wire movement restriction portion switching mechanism, and illustrates a state in which a range of movement of a wire is defined by a moving stopper and a state in which a range of movement of a wire is defined by a fixed stopper;

FIG. 9 is a view that relates to another configuration example of an endoscope apparatus, and illustrates the manner in which switching of a stopper portion is performed in synchrony with an operational input at a switching lever and in which an inside guide sheath is switched between a fixed and non-fixed states by switching a switch;

FIG. 10A is a view illustrating a configuration example in which switching of a stopper portion is performed by rotation of a motor shaft, and a configuration example in which switching of a stopper portion is performed by an advancing/retreating movement of a motor shaft; and

FIG. 10B is a view illustrating a configuration example in which switching of a stopper portion is performed by rotation of a motor shaft, and a configuration example in which switching of a stopper portion is performed by an advancing/retreating movement of a motor shaft.

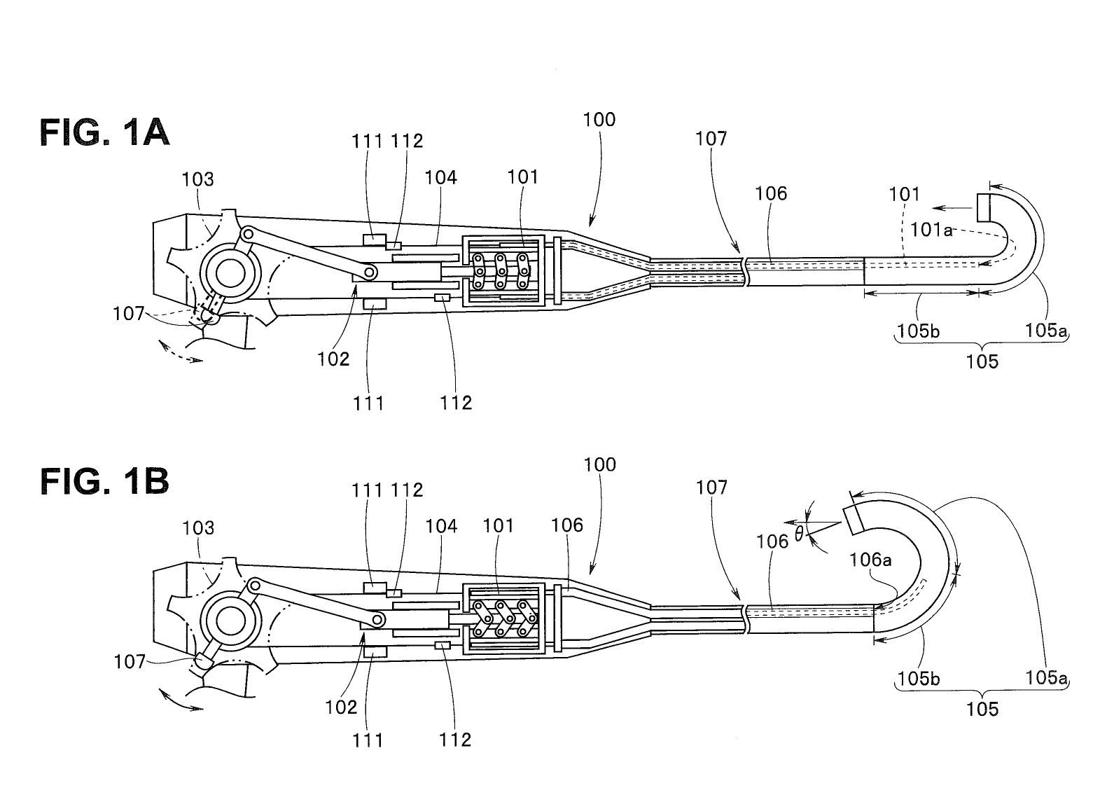

### DETAILED DESCRIPTION OF THE PREFERRED EMBODIMENTS

Embodiments of the present invention are described hereunder with reference to the accompanying drawings.

Note that the drawings are schematic ones in which the relationship between the thickness and width of each member, the thickness ratios of the members and the like are different from those of actual members. Naturally, the drawings include portions in which the dimensional relationships and ratios are different from one another.

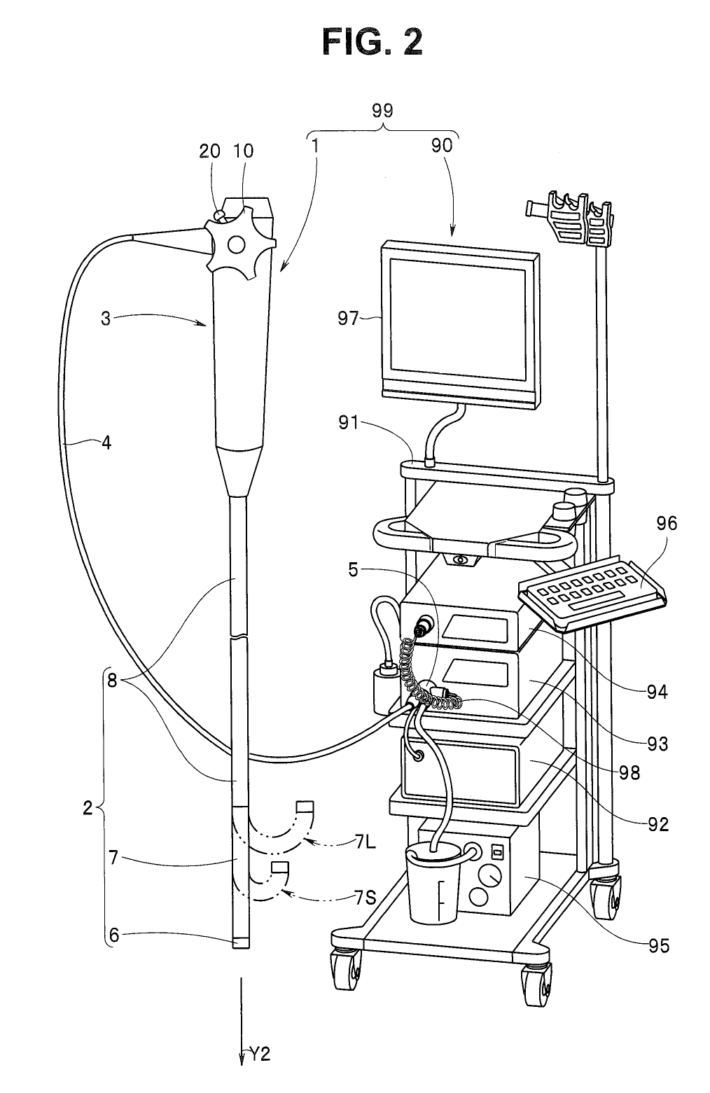

As shown in FIG. 2, an endoscope apparatus 99 includes an endoscope 1 and peripheral apparatuses 90.

The peripheral apparatuses 90 are a control apparatus 92, a light source apparatus 93, a video processor 94, a water feeding apparatus 95, a keyboard 96, and a monitor 97 and the like that are mounted on a plurality of tables provided in a rack 91.

The control apparatus 92 controls lighting of the light source apparatus 93, controls water feeding to the endoscope 1 by the water feeding apparatus 95, and controls image processing by the video processor 94 of electrical signals of a subject image transmitted from an image pickup device of the endoscope 1 as well as outputting of processed images onto a screen of the monitor 97 and the like.

The endoscope 1 is an insertion instrument and includes an insertion portion 2 that is an insertion instrument that is to be inserted into a subject, an operation portion 3 and a universal cord 4. Reference numeral 5 denotes an endoscope connector. The endoscope connector 5 is provided at an end portion of the universal cord 4 that extends from the operation portion 3.

The endoscope connector 5 is detachably connected to the light source apparatus 93. The endoscope connector 5 is electrically connected to the video processor 94 by an optical cable 98.

An upward/downward bending operation knob 10, an air/water feeding operation button (not shown), a suction operation button (not shown), a treatment instrument insertion port (not shown), and a switching lever 21 are provided on the operation portion 3. The upward/downward bending operation knob 10 is a bending operation apparatus.

The insertion portion 2 is formed in an elongated shape, and is constituted by a distal end portion 6, a bending portion 7 and a flexible tube portion 8 that are connected in that order from the distal end side. An illuminating window (not shown), an observation window (not shown), a nozzle (not shown) and a distal end opening (not shown) are provided in the distal end face of the distal end portion 6. The illuminating window is included in an illumination optical system that illuminates a site to be examined. The observation window is included in an image pickup optical system that picks up an image of a site to be examined that is illuminated. The nozzle ejects a fluid for removing body fluids or the like that are attached to the observation window or illuminating window. The distal end opening is an opening on the distal end side of a treatment instrument insertion channel (not shown).

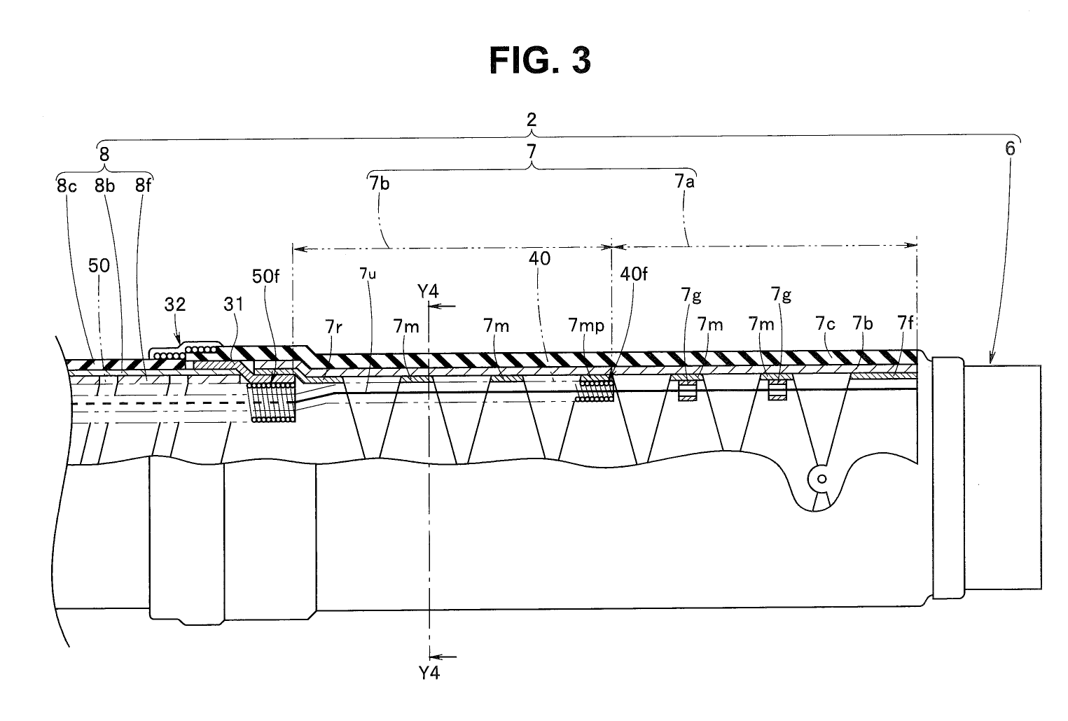

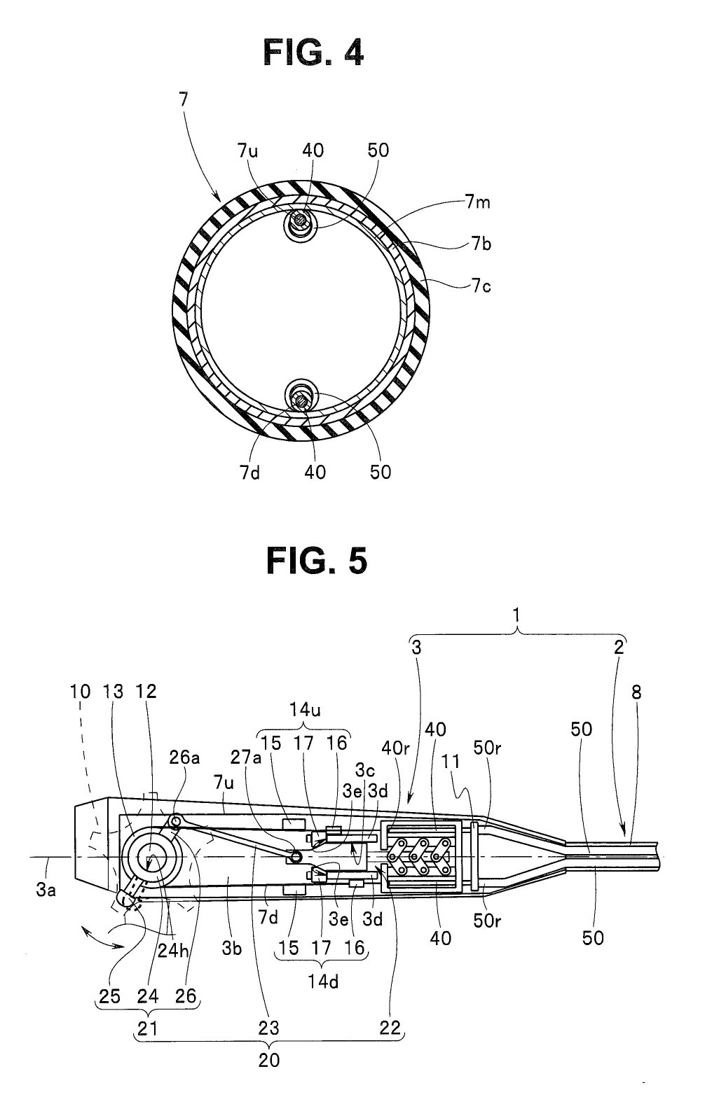

The configuration of the bending portion 7 and of the flexible tube portion 8 will now be described referring to FIG. 3 and FIG. 4.

As shown in FIG. 3 and FIG. 4, two bending wires 7u and 7d are inserted through the inside of the bending portion 7. The bending wires 7u and 7d are provided at positions that correspond to, for example, the upward and downward directions of the endoscope 1.

The bending portion 7 includes a bending piece set. The bending piece set includes a distal end bending piece 7f, a plurality of intermediate bending pieces 7m and a proximal end bending piece 7r that are rotatably connected and configured to be rotatable in the upward/downward directions. The outer circumference of the bending piece set is covered by a braid 7e that is a mesh tube made of metal. The outer circumference of the braid 7e is covered by a bending tube 7c. The bending tube 7c is, for example, a tube made of a rubber such as fluorocarbon rubber.

Reference character 7mp denotes a pipe-fixed bending piece that is one of the intermediate bending pieces 7m. The bending portion 7 includes a first bending region 7a and a second bending region 7b. The first bending region 7a is the distal end side of the bending portion 7, and is a region from the distal end bending piece 7f to the pipe-fixed bending piece 7mp. The second bending region 7b is the proximal end side of the bending portion 7, and is a region from the pipe-fixed bending piece 7mp to the proximal end bending piece 7r.

The respective distal ends of the bending wires 7u and 7d are fixed at previously determined positions that correspond to the upper and lower portions of the distal end bending piece 7f. The bending wires 7u and 7d are inserted through the inside of a wire guide 7g and an inside guide sheath 40, respectively, and extended into the operation portion 3. The bending wires 7u and 7d freely move in the longitudinal axis direction inside the respective wire guides 7g and inside guide sheaths 40.

The respective wire guides 7g are bonded and fixed by solder or the like at positions that correspond to the upper part and lower part, respectively, of the inner circumferential face of the intermediate bending pieces 7m. The distal end portions 40f of the respective inside guide sheaths 40 are bonded and fixed by solder or the like at positions that correspond to the upper part and lower part, respectively, of the inner circumferential face of the pipe-fixed bending piece 7mp.

A proximal end portion of each inside guide sheath 40 (see FIG. 5) is inserted through the inside of an outside guide sheath 50 and guided into the operation portion 3. Each inside guide sheath 40 is capable of advancing and retreating in the longitudinal axis direction within the corresponding outside guide sheath 50.

Distal end portions 50f of the respective outside guide sheaths 50 are bonded and fixed by solder or the like at positions that correspond to the upper part and lower part, respectively, of an inner face on the distal end side of a distal-end-side connecting member that is described later (see reference numeral 31). A proximal end portion 50r (see FIG. 5) of each outside guide sheath 50 is bonded and fixed by solder to a pipe fixing portion (see reference numeral 11 in FIG. 5) that is integrated into an operation portion frame member (reference character 3b in FIG. 5, hereunder abbreviated as “base plate”), which is not shown in the drawings, that is fixedly installed within the operation portion 3.

Note that the inside guide sheaths 40 and the outside guide sheaths 50 are pipe-shaped members that have flexibility, and in the present embodiment the inside guide sheaths 40 and the outside guide sheaths 50 are coil pipes. When the inside guide sheaths 40 and the outside guide sheaths 50 are in a natural state, a loss of bendability of the bending portion 7 and of flexibility of the flexible tube portion 8 is prevented. The bending portion 7 bends accompanying operation of the upward/downward bending operation knob 10. The bending portion 7 has a configuration such that a short bending state 7S and a long bending state 7L that are indicated by chain double-dashed lines in FIG. 2 are selectively obtained accompanying a switching operation of the switching lever 21.

The bending portion 7 is not limited to a configuration that bends in the two directions of upward and downward, and may have a configuration that bends in four directions, namely, upward, downward, left and right. In the case of a configuration that bends in four directions, a bending portion set is configured for the upward, downward, left and right directions, and a bending wire, a wire guide and a guide sheath and the like that correspond to the left and right directions, respectively, are additionally provided.

The flexible tube portion 8 has a predetermined elastic force. More specifically, the flexible tube portion 8 is constituted by a helical tube 8f, a braid 8b and a coating layer 8c. The helical tube 8f is the core element, and is made from a steel plate. The braid 8b covers the outer circumference of the helical tube 8f. The coating layer 8c covers the outer circumference of the braid 8b. The coating layer 8c is provided by heating polyurethane or a polyester-based elastomer and covering the outer circumference of the braid 8b therewith.

The flexible tube portion 8 and the bending portion 7 are connected and fixed to each other through the distal-end-side connecting member 31. The proximal end side of the proximal end bending piece 7r included in the bending piece set is fixed to the outer circumference on the distal end side of the distal-end-side connecting member 31. The distal end side of the braid 8b that is included in the flexible tube portion 8 is fixed to the inner circumference on the proximal end side of the distal-end-side connecting member 31.

The entire length of the distal-end-side connecting member 31 is covered by the bending tube 7c. Reference numeral 32 denotes a bobbin adhesive portion. The bobbin adhesive portion 32 securely fixes the proximal end portion of the bending tube 7c and the distal end portion of the coating layer 8c, and also maintains watertightness between the proximal end face of the bending tube 7c and the distal end face of the coating layer 8c.

The proximal end portion of the flexible tube portion 8 is connected and fixed (the connecting portion is not shown in the drawings) to a distal end portion of the base plate 3b that is fixed to the operation portion 3. The connecting portion is covered by a bend preventing tube (not shown).

As shown in FIG. 5, the outside guide sheaths 50 are fixed to the pipe fixing portion 11 that is provided in the base plate 3b of the operation portion 3. A proximal end portion 40r of each of the inside guide sheaths 40 is extended by a predetermined amount from a proximal end opening of the corresponding outside guide sheath 50.

The proximal end side of the upward bending wire 7u and the proximal end side of the downward bending wire 7d extend from the proximal end opening of the respective inside guide sheaths 40. The proximal end of the upward bending wire 7u is fixed to one end part of a chain (not shown). The chain intermeshes with a sprocket 13 that is integrally connected with and fixed to a shaft 12 of the upward/downward bending operation knob 10. The proximal end of the downward bending wire 7d is fixed to the other end part of the chain (not shown) that intermeshes with the sprocket 13 as described above.

According to this configuration, the chain moves when the upward/downward bending operation knob 10 is rotationally operated in the clockwise direction or counterclockwise direction around its own axis to perform input of an operation. Accompanying movement of the chain, the bending wires 7u and 7d are pulled or slackened and the bending portion 7 performs a bending operation.

Note that, in the above described embodiment the upward bending wire 7u and the downward bending wire 7d are taken as bending wires that are two different wires. However, a configuration may also be adopted in which, for example, the upward bending wire 7u and the downward bending wire 7d are obtained by winding one of the two wires around a pulley (not shown).

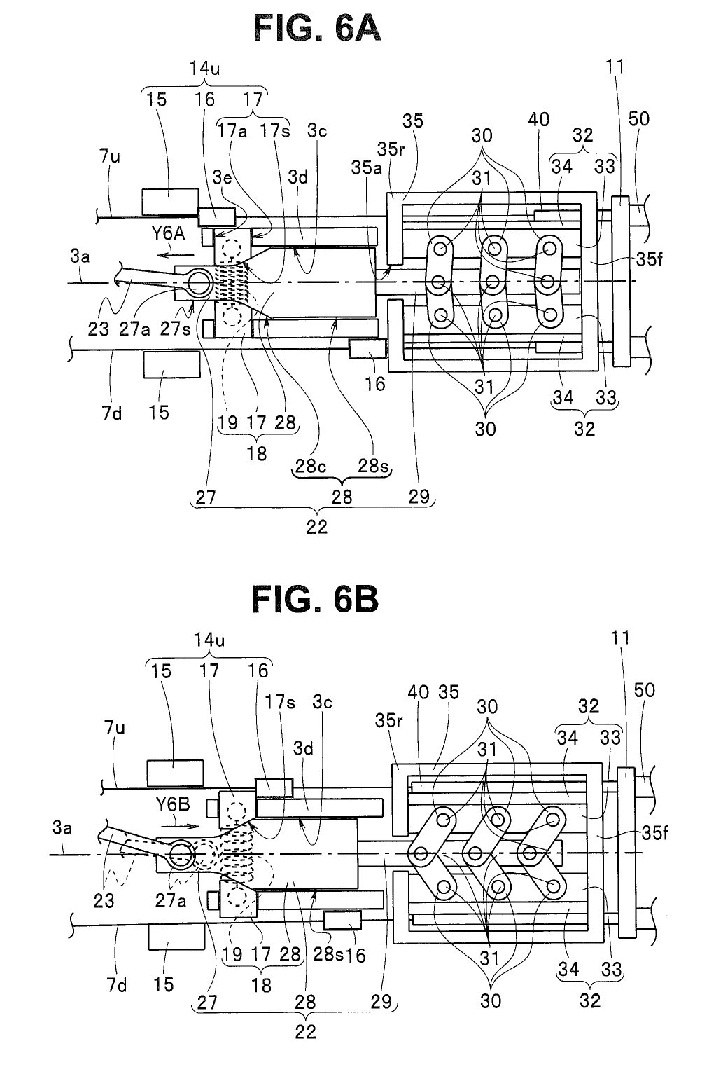

Wire movement restriction portions 14u and 14d, a wire movement restriction portion switching mechanism 18 and a bending-portion bending state switching mechanism 20 that are provided in the operation portion 3 will now be described referring to FIG. 5, FIG. 6A and FIG. 6B.

The bending-portion bending state switching mechanism 20 is a mechanism that switches the proximal end portion 40r of each inside guide sheath 40 to a fixed state or a non-fixed state.

In the non-fixed state, the bending portion 7 is in a first bending state, that is, a long bending state 7L. In the non-fixed state of the bending portion 7, the first bending region 7a and the second bending region 7b of the bending portion 7 perforin a bending operation accompanying a rotational operation of the upward/downward bending operation knob 10.

On the other hand, in the fixed state, the bending portion 7 is in a second bending state, that is, a short bending state 7S. In the fixed state of the bending portion 7, only the first bending region 7a of the bending portion 7 performs a bending operation accompanying a rotational operation of the upward/downward bending operation knob 10.

In the present embodiment, the bending-portion bending state switching mechanism 20 also serves as the wire movement restriction portion switching mechanism 18 that is described later.

First, the wire movement restriction portions 14u and 14d will be described.

Reference character 14u denotes an upward-bending-wire movement restriction portion that restricts movement of the upward bending wire 7u. Reference character 14d denotes a downward-bending-wire movement restriction portion that restricts movement of the downward bending wire 7d.

The wire movement restriction portions 14u and 14d are each constituted by a fixed stopper 15, a stopper contact member 16 and a wire movement range switching stopper (hereunder, abbreviated to “moving stopper”) 17.

The fixed stoppers 15 are fixedly installed at predetermined locations on the base plate 3b. The stopper contact members 16 are fixedly installed at predetermined positions on the wires 7u and 7d.

The moving stoppers 17 are slidably disposed inside a stopper sliding groove 3e, respectively. The stopper sliding grooves 3e are respectively formed at predetermined positions in a pair of wall portions 3d that constitute a switching slide groove 3c that are vertically arranged on the base plate 3b. The position at which the respective moving stoppers 17 are disposed is switched between a first position shown in FIG. 6B and a second position shown in FIG. 6A. At the first position, the respective moving stoppers 17 contact against the contact member 16 and restrict movement of the contact member 16. At the second position, the respective moving stoppers 17 retract from the range of movement of the contact member 16. That is, each moving stopper 17 is configured to be protrudable into and retractable from the range of movement of the stopper contact member 16.

Each moving stopper 17 includes a contact face 17a and an inclined face 17s. The contact face 17a is a face that the stopper contact member 16 comes in contact with. The inclined face 17s is a face that a switching face 28c, described later, of a slide portion main body 28, also described later, comes in contact with.

The switching slide groove 3c is an elongated groove that is formed parallel to an operation portion longitudinal axis 3a. The stopper sliding grooves 3e are formed as grooves that are orthogonal to the operation portion longitudinal axis 3a. Sliding faces 28s of the slide portion main body 28 are slidably disposed in the switching slide groove 3c.

Next, the configuration of the bending-portion bending state switching mechanism 20 will be described.

As shown in FIG. 5, the bending-portion bending state switching mechanism 20 is a link mechanism constituted by a switching lever 21, a slide member 22, and a driving force transmitting member 23. The slide member 22 is disposed so as to be capable of advancing and retreating within the operation portion 3.

The switching lever 21 is rotatably arranged with respect to the shaft 12. The slide member 22 moves forward and rearward along the operation portion longitudinal axis 3a in correspondence with a rotational operation of the switching lever 21. The driving force transmitting member 23 is, for example, an elongated flat plate member. A through-hole that allows one flat face and the other flat face to communicate is provided at both end portions of the driving force transmitting member 23.

The switching lever 21 includes a disc-shaped lever main body 24, an operation protrusion 25 and a connection protrusion 26. The lever main body 24 has a center through-hole 24h that penetrates through one flat face and the other flat face thereof. The center through-hole 24h is disposed on the shaft 12 so that the lever main body 24 is thereby rotatable with respect to the shaft 12.

The operation protrusion 25 is a convex portion that protrudes outward from the outer circumferential face of the lever main body 24. The operation protrusion 25 is operated, for example, by a surgeon who grasps the operation portion 3. The connection protrusion 26 is a convex portion that protrudes outward from the outer circumferential face of the lever main body 24. The connection protrusion 26 is provided at a predetermined position so as to be in a corresponding positional relationship with respect to the operation protrusion 25.

In the present embodiment, a protruding shaft 26a that is a shaft body is provided in a protruding condition from one flat face side of the connection protrusion 26. The protruding shaft 26a is inserted with a predetermined fit into a through-hole that is provided in one end part of the driving force transmitting member 23. Accordingly, the one end part of the driving force transmitting member 23 is pivotally supported in a rotatable manner with respect to the protruding shaft 26a of the switching lever 21.

As shown in FIG. 6A and FIG. 6B, the slide member 22 is constructed by consecutively connecting a connecting portion 27, the slide portion main body 28 and a fixed/non-fixed switching convex portion 29. The connecting portion 27 constitutes a switching lever 21 side of the slide member 22. The fixed/non-fixed switching convex portion 29 constitutes an insertion portion 2 side of the slide member 22.

A connecting shaft 27a that is a shaft body is provided in a protruding condition from one flat face side of the connecting portion 27. The connecting shaft 27a is inserted with a predetermined fit into a through-hole provided in the other end part of the driving force transmitting member 23. Accordingly, the other end part of the driving force transmitting member 23 is pivotally supported in a rotatable manner with respect to the connecting shaft 27a of the slide member 22.

According to the foregoing configurations, a link mechanism is formed such that the slide member 22 moves forward and rearward along the operation portion longitudinal axis 3a accompanying a rotational operation of the switching lever 21.

The slide portion main body 28 also serves as part of the wire movement restriction portion switching mechanism 18. The slide portion main body 28 includes switching faces 28c and sliding faces 28s. The sliding faces 28s are disposed on the insertion portion 2 side of the slide portion main body 28. The sliding faces 28s are one side face and the other side face that slide with respect to inner faces of the wall portions 3d that constitute the switching slide groove 3c.

On the other hand, the switching faces 28c are moving-stopper movement switching portions (abbreviated as “movement switching portion”). The switching faces 28c are an inclined face that links the one side face of the sliding faces 28s and the one side face of the connecting portion 27, and an inclined face that links the other side face of the sliding faces 28s and the other side face of the connecting portion 27.

The moving stoppers 17 are disposed in a condition such that the slide portion main body 28 is interposed therebetween. As a result of the switching faces 28c moving while contacting against the respective inclined faces 17s of the moving stoppers 17, the switching faces 28c impart a force of an amount that causes the moving stoppers 17 to move in a direction that is orthogonal to the movement direction of the slide portion main body 28.

The wire movement restriction portion switching mechanism 18 is constituted by the slide portion main body 28 having the switching faces 28c, the moving stoppers 17 having the inclined faces 17s and, for example, a helical extension spring 19 that is an urging member.

The helical extension spring 19 includes attachment portions that are provided at both end portions thereof. The attachment portions are attached at predetermined positions on the respective moving stoppers 17 that are disposed in a condition such that the slide portion main body 28 is interposed therebetween. The moving stoppers 17 are pulled in the direction of the operation portion longitudinal axis 3a by a tensile force of the helical extension spring 19, and come in contact with or draw close to side faces 27s of the connecting portion 27 and the switching faces 28c. That is, the tensile force of the helical extension spring 19 causes the moving stoppers 17 to be disposed at a second position at which the moving stoppers 17 are retracted from the range of movement of the contact member 16.

One end part of a plurality of switching link members (hereunder, abbreviated as “link member”) 30 that constitute a fixed/non-fixed switching link mechanism is pivotally supported in a rotatable manner in the fixed/non-fixed switching convex portion 29. Through-holes (not shown) for rotatably disposing the one end parts of the link members 30 are arranged in the fixed/non-fixed switching convex portion 29.

Each of the link members 30 is a flat plate member in which a through-hole that allows one flat face and the other flat face thereof to communicate is provided at both end portions thereof. The one end parts of two of the link members 30 are disposed in each through-hole of the fixed/non-fixed switching convex portion 29. These one end parts are rotatably attached to the fixed/non-fixed switching convex portion 29 by, for example, a caulking pin.

Among the two rotatably attached link members 30, the other end part of one of the link members is rotatably attached by means of a caulking pin 31 to a fixed/non-fixed switching slide member (hereunder, abbreviated to “switching slide portion”) 33 that is included in an inside-guide-sheath pressing portion 32 through which the upward bending wire 7u is inserted. The other end part of the other link member is rotatably attached to the switching slide portion 33 that is included in an inside-guide-sheath pressing portion 32 through which the downward bending wire 7d is inserted.

Each inside-guide-sheath pressing portion 32 is constituted by a switching slide portion 33 and a pressing member 34. Through-holes (not shown) through which the caulking pins 31 are inserted are arranged on one side portion side of the respective switching slide portions 33. The pressing member 34 is an elastic member, and is integrally fixed to the other side face of the switching slide portion 33.

Each switching slide portion 33 is disposed inside a fixed/non-fixed switching frame 35 that is constructed in a rectangular shape, and moves forward and rearward along an inner face of the frame in a direction that is orthogonal to the operation portion longitudinal axis 3a. The fixed/non-fixed switching frame 35 is fixedly installed on the base plate 3b.

Note that two through-holes for guide sheaths are formed in an insertion-portion-side wall portion 35f having one sliding face that is an inner frame face of the fixed/non-fixed switching frame 35. The inside guide sheath 40 through which the upward bending wire 7u is inserted is slidably inserted through and disposed inside one of the through-holes for guide sheaths. The inside guide sheath 40 through which the downward bending wire 7d is inserted is slidably inserted through and disposed inside the other of the through-holes for guide sheaths.

Two through-holes for wires, and a switching portion arrangement groove 35a are formed in a switching-lever-side wall portion 35r that has the other sliding face of the fixed/non-fixed switching frame 35. The upward bending wire 7u is slidably inserted through and disposed inside one of the through-holes for wires. The downward bending wire 7d is slidably inserted through and disposed inside the other of the through-holes for wires. The fixed/non-fixed switching convex portion 29 is slidably arranged in the switching portion arrangement groove 35a.

The relations among the switching lever 21, the slide member 22, the moving stoppers 17 and the inside-guide-sheath pressing portions 32 will now be described.

When the operation protrusion 25 of the switching lever 21 is at a position shown by a solid line in FIG. 5, the state is one in which the slide member 22 has moved furthest to the insertion portion 2 side, as shown in FIG. 6A. At this time, the inside-guide-sheath pressing portions 32 are in an inside-guide-sheath fixing state. The pressing member 34 of each of the pressing portions 32 presses against and closely contacts the corresponding inside guide sheath 40. The moving stoppers 17 are disposed at a retracted position that is the second position by the tensile force of the helical extension spring 19.

On the other hand, when the operation protrusion 25 of the switching lever 21 is at a position shown by a dashed line in FIG. 5, the state is one in which the slide member 22 has moved furthest to the switching lever 21 side as shown in FIG. 6B. At this time, the inside-guide-sheath pressing portions 32 are in an inside-guide-sheath non-fixing state. The pressing member 34 of each of the pressing portions 32 is separated from the corresponding inside guide sheath 40. The moving stoppers 17 are disposed at the first position after moving against the urging force of the helical extension spring 19.

The moving stoppers 17 are stably retained at the first position because the switching faces 28c of the slide portion main body 28 are in a state of contact against the inclined faces 17s of the moving stoppers 17.

Subsequently, in the inside-guide-sheath non-fixing state shown in FIG. 6B, the operation protrusion 25 of the switching lever 21 is rotatingly moved in a gradual manner from the position indicated by the dashed line to the position indicated by the solid line in FIG. 5. In response thereto, the slide member 22 moves towards the insertion portion 2 side (moves in the arrow Y6B direction in FIG. 6B).

Accompanying the movement of the slide member 22, the moving stoppers 17 gradually move from the first position towards the second position because of the tensile force of the helical extension spring 19. Further, accompanying the movement of the slide member 22, the pressing members 34 are moved to approach the inside guide sheaths 40 as a result of the pairs of rotatable link members 30 being rotated in a direction that gradually increases an internal angle therebetween by the fixed/non-fixed switching convex portion 29.

Conversely, for example, in the inside-guide-sheath fixing state shown in FIG. 6A, the operation protrusion 25 of the switching lever 21 is rotatingly moved in a gradual manner from the position indicated by the solid line to the position indicated by the dashed line in FIG. 5. In response thereto, the slide member 22 moves towards the switching lever 21 side (in the arrow Y6A direction in FIG. 6A).

Accompanying movement of the slide member 22, the switching faces 28c of the slide portion main body 28 move while contacting against the inclined faces 17s. As a result, the moving stoppers 17 gradually move from the second position towards the first position against the urging force of the helical extension spring 19. Further, accompanying movement of the slide member 22, the respective pressing members 34 are moved in a direction away from the corresponding inside guide sheaths 40 as a result of the pairs of rotatable link members 30 being rotated in a direction that gradually decreases the internal angle therebetween by the fixed/non-fixed switching convex portion 29.

That is, the moving stoppers 17 and the inside-guide-sheath pressing portions 32 synchronously operate in response to and in synchrony with an operational input at the switching lever 21.

As shown in FIG. 6A, when the moving stoppers 17 are disposed at the second position and the inside guide sheaths 40 are in a fixed state, the upward/downward bending operation knob 10 is rotationally operated to, for example, pull the upward bending wire 7u. Thereupon, the stopper contact member 16 contacts against the fixed stopper 15 without contacting against the moving stopper 17 that is disposed at the second position. A state in which the stopper contact member 16 contacts against the fixed stopper 15 is defined as a second-bending-state maximum wire pulling state.

In contrast, as shown in FIG. 6B, when the moving stoppers 17 are disposed at the first position and the inside guide sheaths 40 are in a non-fixed state, the upward/downward bending operation knob 10 is rotationally operated to, for example, pull the upward bending wire 7u. Thereupon, the stopper contact member 16 contacts against the contact face 17a of the moving stopper 17 that is disposed at the first position. A state in which the stopper contact member 16 contacts against the contact face 17a is defined as a first-bending-state maximum wire pulling state.

Note that, in the present embodiment, at the time of the first-bending-state maximum wire pulling state, the bending portion 7 that is in the long bending state 7L shown in FIG. 2 is set so as to bend at, for example, a maximum bending angle of 180° in the upward direction.

On the other hand, at the time of the second-bending-state maximum wire pulling state, the bending portion 7 that is in the short bending state 7S shown in FIG. 2 is set so as to bend at, for example, a maximum bending angle of 180° in the upward direction.

Operation of the endoscope 1 that is configured as described above will now be described.

When using the endoscope 1, for example, a surgeon considers whether to place the bending portion 7 in the first bending state or the second bending state. In a case where the surgeon performs an examination in the first bending state, that is, in a case where the long bending state 7L in which the entire bending portion 7 is bent is obtained, the inside guide sheaths 40 are in the non-fixed state as shown in FIG. 6B. At this time, the moving stoppers 17 are disposed at the first position.

In the first bending state, the surgeon operates the bending operation knob 10 to, for example, perform an operation to bend the bending portion 7 in the upward direction. Thereupon, accompanying pulling of the upward bending wire 7u, the first bending region 7a of the bending portion 7 gradually bends. Accompanying bending of the first bending region 7a, a compressive force along the extension direction acts on the inside guide sheath 40 whose distal end portion 40f is fixed in the second bending region 7b of the bending portion 7. When this compressive force becomes greater than a previously determined amount of force accompanying bending of the first bending region 7a, the proximal end portion 40r of the inside guide sheath 40 that is in the non-fixed state cannot resist the compressive force and consequently moves in the switching lever 21 direction. In contrast, because the distal end portion 50f and the proximal end portion 50r of the outside guide sheath 50 that is inside the flexible tube portion 8 are fixed, the outside guide sheath 50 resists a compressive force that acts along the extension direction of the outside guide sheath 50.

As a result, the entire bending portion 7 performs a bending operation that takes the distal end portion 50f of the outside guide sheath 50 as an origin. Further, the bending portion 7 that is in the long bending state 7L bends at the maximum bending angle of 180° with respect to the upward direction when the stopper contact member 16 contacts against the moving stopper 17.

On the other hand, in a case where the surgeon performs an examination in the second bending state, that is, in a case where the short bending state 7S in which only the first bending region 7a of the bending portion 7 is bent is obtained, the inside guide sheaths 40 are in the fixed state as shown in FIG. 6A. At this time, the moving stoppers 17 are retracted to the second position.

In the second bending state, the surgeon operates the bending operation knob 10 to, for example, perform an operation to bend the bending portion 7 in the upward direction. Thereupon, accompanying pulling of the upward bending wire 7u, the first bending region 7a of the bending portion 7 gradually bends. Accompanying bending of the first bending region 7a, a compressive force along the extension direction acts on the inside guide sheath 40 whose distal end portion 40f is fixed in the second bending region 7b of the bending portion 7. At this time, the proximal end portion 40r of the inside guide sheath 40 is in a fixed state. Consequently, the inside guide sheath 40 resists a compressive force that acts along the extension direction that is applied to the inside guide sheath 40.

As a result, only the first bending region 7a of the bending portion 7 performs a bending operation that takes the distal end portion 40f of the inside guide sheath 40 as an origin. Further, the bending portion 7 that is in the short bending state 7S bends at the maximum bending angle of 180° with respect to the upward direction when the stopper contact member 16 contacts against the fixed stopper 15.

Subsequently, upon the surgeon performing an operation to move the operation protrusion 25 of the switching lever 21, the slide member 22 moves in synchrony with the aforementioned operation, and the moving stoppers 17 and the guide sheath pressing portions 32 operate in response to the movement of the slide member 22.

That is, when the bending portion 7 is switched from, for example, the first bending state to the second bending state, the guide sheath pressing portions 32 are switched from a non-fixing state to a fixing state and the moving stoppers 17 are switched from the first position to the second position.

Conversely, when the bending portion 7 is switched from, for example, the second bending state to the first bending state, the guide sheath pressing portions 32 are switched from the fixing state to the non-fixing state and the moving stoppers 17 are switched from the second position to the first position.

Thus, the switching faces 28c that move while contacting against the inclined faces 17s are provided in the slide member 22 that moves forward or rearward accompanying a rotational operation of the switching lever 21 that is included in the bending-portion bending state switching mechanism 20, and the fixed/non-fixed switching convex portion 29 is provided to which the link members 30 are attached that move the inside-guide-sheath pressing portions 32 that switch the inside guide sheaths to a fixed state or a non-fixed state.

According to this configuration, an operation that switches the proximal end portion of the relevant inside guide sheath 40 to a fixed state or a non-fixed state and an operation that disposes the moving stoppers 17 at the second position or the first position can be performed in response to and in synchrony with an operational input at the switching lever 21.

Further, at a time that the surgeon operates the bending operation knob 10 to obtain a maximum wire pulling state, the maximum bending angle of the bending portion 7 becomes 180° irrespective of whether the state is the first bending state or the second bending state.

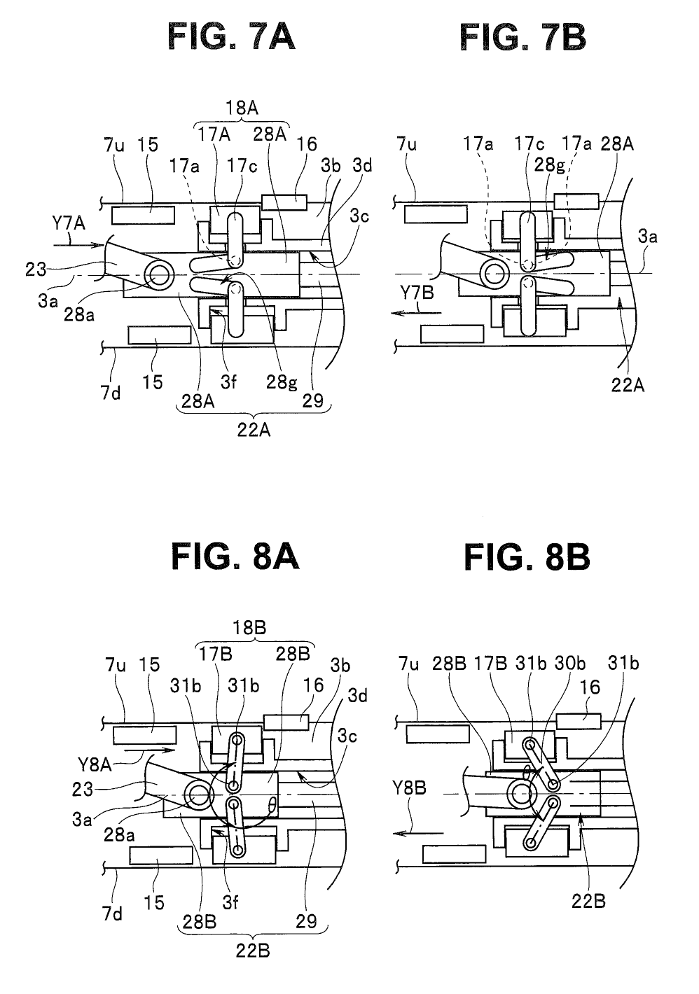

Note that, the configuration of the wire movement restriction portion switching mechanism that switches the wire movement restriction portion in synchrony with an operation that switches the bending state of the bending portion is not limited to the above described configuration, and may be a configuration shown in FIGS. 7A and 7B or FIGS. 8A and 8B or the like.

In the present embodiment as shown in FIGS. 7A and 7B, a wire movement restriction portion switching mechanism 18A is provided inside the operation portion 3. The wire movement restriction portion switching mechanism 18A is constituted by a slide portion main body 28A and moving stoppers 17A.

In the present embodiment, a slide member 22A is constituted by the slide portion main body 28A that also serves as the connecting portion 27, and the fixed/non-fixed switching convex portion 29. The slide portion main body 28A has a pair of cam grooves 28g that are inclined at a predetermined angle relative to the operation portion longitudinal axis 3a. The other end part of the driving force transmitting member 23 is pivotally supported in a rotatable manner with respect to a protruding shaft 28a on the slide portion main body 28A.

Each of the moving stoppers 17A includes a convex portion 17c having an extending portion that protrudes by a predetermined amount from the side face of the relevant stopper. The respective extending portions are orthogonally disposed relative to the operation portion longitudinal axis 3a.

The cam grooves 28g are movement switching portions. A cam shaft 17b that is provided at an extending end portion of each of the convex portions 17c is slidably disposed inside the corresponding cam groove 28g.

The respective moving stoppers 17A are slidably disposed in stopper slide concave portions 3f. The stopper slide concave portions 3f are provided at predetermined positions in the pair of wall portions 3d that constitute the switching slide groove 3c that are vertically arranged on the base plate 3b. The extending portion of each convex portion 17c is slidably disposed inside a concave portion provided in the wall portions 3d.

The remaining configuration is the same as in the above described embodiment, and the same members are denoted by the same reference characters, and hence a description of such members is omitted.

Operation of the wire movement restriction portion switching mechanism 18A will now be described.

In a state in which the moving stoppers 17A are disposed at the first position that is shown in FIG. 7A, the slide member 22A is moved in the direction of an arrow Y7A. Accompanying the movement of the slide member 22A, the cam shafts 17b that are disposed in the cam grooves 28g move relatively from one end side to the other end side of the corresponding cam groove 28g. At this time, an amount of force that causes the cam shafts 17b to gradually move in a direction that is orthogonal to the direction of the operation portion longitudinal axis 3a is imparted from each of the cam grooves 28g to the corresponding cam shafts 17b. As a result, the moving stoppers 17A are gradually moved inside the stopper slide concave portions 3f to approach the operation portion longitudinal axis 3a, and are thus switched to and disposed at the second position.

On the other hand, in a state in which the moving stoppers 17A are disposed at the second position that is shown in FIG. 7B, the slide member 22A is moved in the direction of an arrow Y7B. Accompanying the movement of the slide member 22A, the cam shafts 17b that are disposed in the cam grooves 28g move relatively from the other end side to the one end side of the corresponding cam groove 28g. At this time, an amount of force that causes the cam shafts 17b to gradually move in a direction that is orthogonal to the direction of the operation portion longitudinal axis 3a is imparted from each of the cam grooves 28g to the corresponding cam shafts 17b. As a result, the moving stoppers 17A are gradually moved inside the stopper slide concave portions 3f so as to move away from the operation portion longitudinal axis 3a and arrive at the first position, and are thus switched to and disposed at the first position.

That is, in the present embodiment, similarly to the above described embodiment, the moving stoppers 17A can be made protrudable into and retractable from the range of movement of the stopper contact members 16 accompanying forward or rearward movement of the slide member 22A.

Consequently, similar operations and effects as those in the above described embodiment can be obtained.

Further, in the present embodiment, a configuration is adopted in which the moving stoppers 17A are switched to the first position or the second position by forward or rearward movement of the slide member 22A. Accordingly, the helical extension spring 19 can be dispensed with. Consequently, when moving the slide member 22A by operating the switching lever 21, a lever operation to cause the slide member 22A to move against an urging force of a spring is eliminated, and thus the operability of the lever is improved.

In the present embodiment as shown in FIGS. 8A and 8B, a wire movement restriction portion switching mechanism 18B is provided inside the operation portion 3. The wire movement restriction portion switching mechanism 18B is constituted by a slide portion main body 28B and moving stoppers 17B.

A slide member 22B of the present embodiment is constituted by the slide portion main body 28B that also serves as the connecting portion 27, and the fixed/non-fixed switching convex portion 29. The other end part of the driving force transmitting member 23 is pivotally supported in a rotatable manner with respect to the protruding shaft 28a on the slide portion main body 28B. In addition, end parts of respective link members 30b are rotatably attached to the slide portion main body 28B by caulking pins 31b. The link members 30b are movement switching portions.

The other end part of the respective link members 30b are rotatably attached to the moving stoppers 17B by caulking pins 31b. The moving stoppers 17B are slidably disposed in the corresponding stopper slide concave portions 3f. The stopper slide concave portions 3f are provided at predetermined positions in the pair of wall portions 3d that constitute the switching slide groove 3c that are vertically arranged on the base plate 3b. Each link member 30b is movably disposed inside an elongated concave portion provided in the corresponding wall portion 3d.

The remaining configuration is the same as in the above described embodiment, and the same members are denoted by the same reference characters, and hence a description of such members is omitted.

Operation of the wire movement restriction portion switching mechanism 18B will now be described.

In a state in which the moving stoppers 17B are disposed at the first position that is shown in FIG. 8A, the slide member 22B is moved in the direction of an arrow Y8A. Accompanying the movement of the slide member 22B, the two link members 30b each rotate and an angle 8 that is formed by the two link members 30b gradually decreases. As a result, the moving stoppers 17B are gradually moved in a direction that is orthogonal to the direction of the operation portion longitudinal axis 3a by a force that is imparted from the link members 30b. Specifically, the moving stoppers 17B are moved within the stopper slide concave portions 3f so as to approach the operation portion longitudinal axis 3a and thus arrive at the second position.

On the other hand, in a state in which the moving stoppers 17B are disposed at the second position that is shown in FIG. 8B, the slide member 22B is moved in the direction of an arrow Y8B. Accompanying the movement of the slide member 22B, the two link members 30b each rotate and an angle θ that is formed by the two link members 30b gradually increases. As a result, the moving stoppers 17B are gradually moved in a direction that is orthogonal to the direction of the operation portion longitudinal axis 3a by a force that is imparted from the link members 30b. Specifically, the moving stoppers 17B are moved within the stopper slide concave portions 3f so as to move away from the operation portion longitudinal axis 3a and thus arrive at the first position.

That is, in the present embodiment also, similarly to the above described embodiment, the moving stoppers 17B can be made protrudable into and retractable from the range of movement of the stopper contact members 16 accompanying forward or rearward movement of the slide member 22B.

Consequently, similar operations and effects as those in the above described embodiment can be obtained.

Note that the configurations of the wire movement restriction portion switching mechanisms 18, 18A and 18B described with reference to FIG. 6A, FIG. 6B, FIG. 7A, FIG. 7B, FIG. 8A and FIG. 8B are examples, and other configurations may also be adopted to cause the moving stoppers 17, 17A or 17B and the inside-guide-sheath pressing portions 32 to move in response to and in synchrony with an operational input at the switching lever 21.

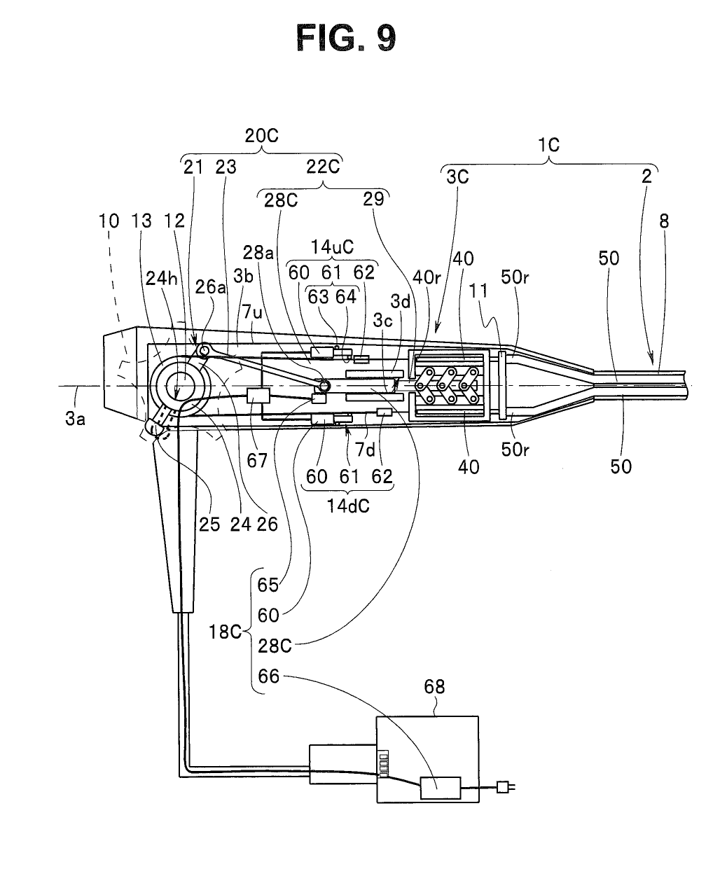

A configuration will now be described with reference to FIG. 9 in which, by use of an electrical switch, switching of positions at which stopper portions 63 and 64, described later, are disposed and fixing/non-fixing by means of the guide sheath pressing portions 32 is performed in response to and in synchrony with an operational input at the switching lever 21.

An endoscope 1C shown in FIG. 9 includes wire movement restriction portions 14uC and 14dC, a wire movement restriction portion switching mechanism 18C and a bending-portion bending state switching mechanism 20C in an operation portion 3C.

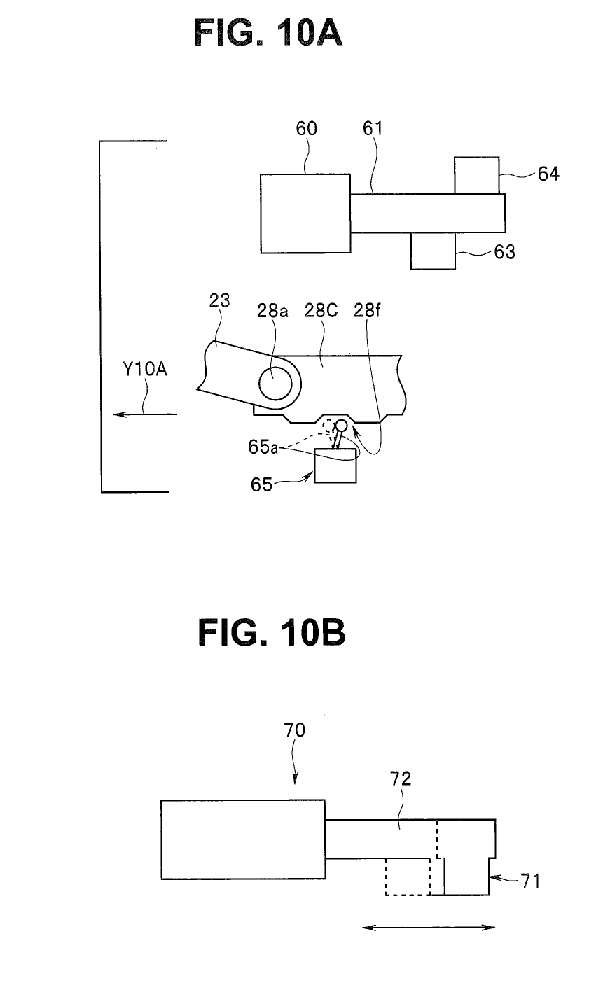

In the present embodiment, as shown in FIG. 9 and FIG. 10A, the wire movement restriction portions 14uC and 14dC are constituted by a motor 60, a motor shaft 61 and a stopper contact member 62. A first stopper portion 63 and a second stopper portion 64 are integrally provided on the motor shaft 61. The motor 60 is fixedly installed at a predetermined location on the base plate 3b.

Similarly to the above described fixed stopper, the first stopper portion 63 is a stopper that defines the second-bending-state maximum wire pulling state by the stopper contact member 62 coming in contact with the first stopper portion 63. On the other hand, the second stopper portion 64 is a stopper that defines the first-bending-state maximum wire pulling state by the stopper contact member 62 coming in contact with the second stopper portion 64. The endoscope 1C is configured so that one of the first stopper portion 63 and the second stopper portion 64 is disposed in the range of movement of the stopper contact member 62 as a result of the motor shaft 61 being rotated by 180°.

Next, the configuration of the bending-portion bending state switching mechanism 20C will be described.

The bending-portion bending state switching mechanism 20C is a link mechanism that is constituted by the switching lever 21, a slide member 22C that is disposed so as to be capable of advancing and retreating within the operation portion 3, and the driving force transmitting member 23. In the present embodiment, the slide member 22C is constituted by connecting a slide portion main body 28C that is integrated with the connecting portion 27, and the fixed/non-fixed switching convex portion 29. The protruding shaft 28a is provided on the slide portion main body 28C, and the other end part of the driving force transmitting member 23 is pivotally supported in a rotatable manner on the protruding shaft 28a.

The slide portion main body 28C also serves as a part of the wire movement restriction portion switching mechanism 18C. One side face of the slide portion main body 28C is configured as a switch-switching face 28f. The switch-switching face 28f is configured so as to switch a lever switch 65a of a micro switch 65.

The wire movement restriction portion switching mechanism 18C is constituted by the slide portion main body 28C, the micro switch 65, the motor 60 and the control portion 66. The slide portion main body 28C has the switch-switching face 28f. The micro switch 65 is, for example, a lever-type switching switch.

Reference numeral 67 denotes a connection substrate. A signal wire and the like that extend from the respective motors 60 for upward and downward bending as well as a signal wire and the like that extend from the micro switch 65 are electrically connected to the connection substrate 67. In addition, a signal wire that extends from a control portion 66 provided inside a light source apparatus 68 that is an external apparatus is electrically connected to the connection substrate 67. Note that a configuration may also be adopted in which signal wires and the like are directly connected to the control portion 66 without providing the connection substrate 67.

In the present embodiment, a signal is outputted from the micro switch 65, the output signal is inputted to the control portion 66, the control portion 66 outputs an instruction signal to the motor 60, the motor shaft 61 of the motor 60 is rotated, and one of the first stopper portion 63 and the second stopper portion 64 that is rotated 180° is disposed in the range of movement of the stopper contact member 62.

The remaining configuration is the same as in the above described embodiment, and the same members are denoted by the same reference symbols, and hence a description of such members is omitted.

Note that, as shown in FIG. 10A, as a result of the slide portion main body 28C moving in the direction of an arrow Y10A, the lever switch 65a of the micro switch 65 is switched from a state in which the lever switch 65a is tilted to the right side that is indicated by a solid line in FIG. 10A to a state in which the lever switch 65a is tilted to the left side that is indicated by a dashed line in FIG. 10A. When the lever switch 65a is in the state indicated by the solid line, the first stopper portion 63 is disposed within the range of movement of the stopper contact member 62. Further, the inside-guide-sheath pressing portion 32 is in the inside-guide-sheath fixing state in which the inside-guide-sheath pressing portion 32 presses against the inside guide sheath 40.

The relations among the switching lever 21, the slide member 22C, the wire movement restriction portion switching mechanism 18C and the inside-guide-sheath pressing portion 32 will now be described.

The switching lever 21 is operated to cause the slide member 22C to move towards the switching lever 21 side (direction of an arrow Y10A in FIG. 10A). Thereupon, along with the lever switch 65a being switched to the tilted state that is indicated by the dashed line, the inside-guide-sheath fixing state is switched to the non-fixing state by the inside-guide-sheath pressing portion 32.

Upon the tilting direction of the micro switch 65 being switched, a first output signal is outputted from the switch 65 to the control portion 66. Upon receiving the first output signal, the control portion 66 outputs a first instruction signal to the motor 60. Upon receiving the first instruction signal, the motor 60 rotates the motor shaft 61 by 180° and thus the second stopper portion 64 is switched in place of the first stopper portion 63 so as to be disposed within the range of movement of the stopper contact member 62.

On the other hand, the switching lever 21 is operated from the above described state to cause the slide member 22C to move in the opposite direction. Thereupon, along with the lever switch 65a being switched to the tilted state that is indicated by the solid line from the tilted state that is indicated by the dashed line, the inside-guide-sheath non-fixing state is switched to the fixing state.

At this time, a second output signal is outputted from the switch 65 to the control portion 66. Upon receiving the second output signal, the control portion 66 outputs a second instruction signal to the motor 60. Upon receiving the second instruction signal, the motor 60 rotates the motor shaft 61 by 180° and thus the first stopper portion 63 is switched in place of the second stopper portion 64 so as to be disposed within the range of movement of the stopper contact member 62.

That is, in the present embodiment, the motor shaft 61 that has the stopper portions 63 and 64, and the inside-guide-sheath pressing portion 32 move in response to and in synchrony with an operational input at the switching lever 21. That is, by switching the tilting direction of the lever switch 65a by means of the switch-switching face 28f of the slide portion main body 28C, the motor shaft 61 is rotated and similar operations and effects as those in the above described embodiment can be obtained.

Note that the switching switch is not limited to the above described lever-type micro switch, and may be a push-button type switch or a toggle switch or the like. A configuration may also be adopted so as to press a push-button type switch with an end face on a lever side of the slide portion main body 28B. Further, the switching switch may be a non-contact switch that uses an optical sensor or a proximity sensor.

Further, in the above described embodiment, a configuration is adopted in which the motor shaft 61 is rotated so as to switchingly dispose the first stopper portion 63 and the second stopper portion 64 in the range of movement of the stopper contact member 62. However, a linear actuator 70 that is shown in FIG. 10B or a solenoid that is not shown in the drawings may also be used.

The linear actuator 70 has, for example, an L-shaped shaft portion 72 that includes a stopper portion 71. The shaft portion 72 of the linear actuator 70 is capable of advancing and retreating in the operation portion longitudinal axis 3a direction, and the stopper portion 71 advances and retreats within the range of movement of the stopper contact member 62.

In the present embodiment, the shaft portion 72 changes between a first protruding state that is indicated by a solid line and a second protruding state that is indicated by a dashed line. In the first protruding state the first-bending-state maximum wire pulling state is obtained, and in the second protruding state the second-bending-state maximum wire pulling state is obtained.

In the above described embodiment, a configuration is adopted in which the switch is switched by movement of the slide member 22C. However, a position at which the switching switch is disposed is not limited to the vicinity of the slide member 22C that is included in the link mechanism. A configuration may also be adopted in which the position at which the switching switch is disposed is in the vicinity of the driving force transmitting member 23 that is included in the link mechanism, the vicinity of the lever main body 24 of the switching lever 21, or the vicinity of the operation protrusion 25 or the like.

Further, in the configuration including the fixed stopper 15 that is shown in FIGS. 6A and 6B, the respective moving stoppers may be configured so as to be caused to protrude into or retract from the range of movement of the stopper contact member 62 by the driving force of a driving apparatus such as a motor. For example, a configuration may be adopted in which the moving stoppers protrude or retract in the vertical direction from one face of the base plate 3b.

In the above described embodiments, the insertion instrument is described as an endoscope. However, the insertion instrument is not limited to an endoscope, and as long as the configuration includes a bending portion having a first bending region and a second bending region in an insertion portion, the insertion instrument is also applicable to a guide tube, various kinds of treatment instruments and a manipulator or the like.

It should be understood that the present invention is not limited only to the above described embodiments, and various modifications can be made without departing from the spirit and scope of the invention.

What is claimed is:

1. An insertion instrument, comprising:
an elongated insertion portion to be inserted into a subject;
a bending portion that is bendably provided on a distal end side of the insertion portion and that has a first bending region and a second bending region;
a wire that is inserted through inside of the insertion portion and that is capable of performing an advancing movement and a retreating movement along the insertion portion to cause the bending portion to perform a bending operation;
a bending state switching mechanism that switches between a first bending state in which the first bending region and the second bending region of the bending portion perform a bending operation and a second bending state in which only the first bending region performs a bending operation;
a restriction portion that restricts a range in which the wire moves in the first bending state or the second bending state; and
a restriction portion switching mechanism that, in response to an operation that switches to the first bending state or the second bending state by the bending state switching mechanism, switches a range in which the wire moves by means of the restriction portion.

2. The insertion instrument according to claim 1, wherein, by switching the restriction portion by means of the restriction portion switching mechanism, a range in which the wire moves at a time of the first bending state is set to a shorter range than a range in which the wire moves at a time of the second bending state, and a maximum bending angle at a time of the first bending state and a maximum bending angle at a time of the second bending state are set to a same angle.

3. The insertion instrument according to claim 2, wherein:
the restriction portion switching mechanism comprises a fixed stopper that is fixedly installed in an integrated manner in an operation portion that is connected to a proximal end side in an insertion direction of the insertion portion, and a moving stopper that is protrudable into and retractable from a range in which a stopper contact member that is fixed to the wire moves; and
the moving stopper is disposed at a first position at which the stopper contact member comes in contact with the moving stopper or at a second position at which the moving stopper is retracted from a range of movement of the stopper contact member by a slide member that comprises a bending state switching mechanism that is provided in the operation portion and that moves forward or rearward accompanying an operation of a switching lever of the bending state switching mechanism.

4. The insertion instrument according to claim 3, wherein, accompanying movement of a slide portion main body that comprises the slide member, the moving stopper is moved in a direction that is orthogonal to a movement direction of the slide member.

5. The insertion instrument according to claim 3, wherein:
the restriction portion switching mechanism further comprises a driving apparatus that causes the moving stopper to protrude or retract; and
the driving apparatus causes the moving stopper to protrude into or retract from the range of movement of the stopper contact member based on an output signal that is outputted from a switching switch that is switchingly operated by forward or rearward movement of a slide portion main body comprising the slide member.

6. The insertion instrument according to claim 2, wherein:
the restriction portion switching mechanism comprises: a driving apparatus comprising a stopper portion; an operation switch that outputs an output signal for switching a position of the stopper portion; and a control portion that is connected to the operation switch and the driving apparatus and that outputs to the driving apparatus an instruction signal that is based on the output signal; and
the operation switch is disposed in a vicinity of a member comprising a link mechanism of the bending state switching mechanism.

7. The insertion instrument according to claim 6, wherein:
the driving apparatus comprises a first stopper portion and a second stopper portion on a drive shaft, and by the drive shaft rotating 180°, one of the stopper portions is disposed in a range of movement of a stopper contact member that is fixed to the wire.

8. The insertion instrument according to claim 6, wherein:
the driving apparatus comprises a stopper portion on a drive shaft, and by the drive shaft moving forward or rearward in a longitudinal axis direction of an operation portion, a position at which the stopper portion is disposed is switched to a position corresponding to the first bending state or a position corresponding to the second bending state.

9. An endoscope having a configuration of an insertion instrument according to claim 1.
